(19) United States
(12) Patent Application Publication
(10) Pub. No.: US 2026/0265740 A1
(43) Pub. Date: Sep. 10, 2026

(54) COMPOSITION FOR PREVENTING OR TREATING CEREBRAL INFARCTION

(71) Applicants: JEONNAM BIOINDUSTRY FOUNDATION, Jeollanam-do (KR); INDUSTRY FOUNDATION OF CHONNAM NATIONAL UNIVERSITY, Gwangju (KR)

(72) Inventors: HAN SEONG JEONG, Jeollanam-do (KR); SU JEONG JANG, Jeollanam-do (KR); EUNG PIL KIM, Jeollanam-do (KR); EUN JAE JANG, Jeollanam-do (KR); Hee YU, Jeollanam-do (KR)

(21) Appl. No.: 19/165,271

(22) PCT Filed: Feb. 27, 2024
(86) PCT No.: PCT/KR2024/002498

§ 371 Date: Sep. 15, 2025

(30) Foreign Application Priority Data
Mar. 15, 2023 (KR) 10-2023-0034223

(51) Int. Cl. C12N 15/113; A61K 31/7105; A61P 9/10

(52) U.S. Cl. CPC C12N 15/113; A61K 31/7105; A61P 9/10; C12N 2310/141; C12N 2320/30

(57) ABSTRACT

A method for treating cerebral infarction includes administering a composition including micro RNA-382-5P (miR-382-5P) to a subject in need thereof. The miR-382-5P is involved in interneuronal adhesion, nervous system development, vocalization behavior, neurotrophin signaling pathways, dopaminergic synapses, PI3K-AKT signaling pathways, and apoptosis. The composition has an excellent effect in reducing the extent or severity of cerebral infarction.

### REFERENCE TO AN ELECTRONIC SEQUENCE LISTING

A sequence listing electronically submitted on Sep. 15, 2025 as a XML file named 20250915_LC1212515_TU_SEQ.XML, created on Sep. 12, 2025 and having a size of 1,517 bytes, is incorporated herein by reference in its entirety.

### BACKGROUND

### 1. Technical Field

The present invention relates to a pharmaceutical composition and a health functional food for preventing or treating cerebral infarction.

### 2. Background Art

Ontogenesis proceeds through a developmental regulation process of a fertilized egg. In tissue formation, cells forming a tissue are cells with specific types and functions, and these cells are derived from undifferentiated cells. Stem cells are relatively undifferentiated cells that have not yet developed and have the ability to differentiate into specific tissue cells. These cells exist during the developmental process and are not defined in situations where artificial regulation is applied. In other words, stem cells have the characteristics of relatively undifferentiated cells at each developmental stage. They also have the characteristics of simultaneously producing daughter cells that have the ability to maintain their own characteristics and daughter cells that differentiate into specific types of cells.

Embryonic stem cells are stem cells (pluripotent stem cells) that have the ability to differentiate into all types of cells that make up an organism. Other stem cells can only differentiate into specific tissue cells.

Cerebral infarction refers to a neurological disorder that occurs when a cerebral blood vessel is suddenly blocked. On the other hand, cerebral hemorrhage refers to a disease in which a cerebral blood vessel bursts. Stroke is a common term referring to both cerebral infarction (ischemic stroke) and cerebral hemorrhage (hemorrhagic stroke).

Cerebral infarction is caused by arteriosclerosis, heart disease, cerebrovascular malformation, and vascular dissection. Therefore, being aged 60 years or older, diabetes, high blood pressure, hyperlipidemia (patients with high cholesterol), smoking, drinking, and obesity are risk factors for cerebral infarction because they are causes of arteriosclerosis. Further, embolic cerebral infarction may occur due to heart disease (myocardial infarction, arrhythmia, valvular disease), and taking birth control pills is also a risk factor for cerebral infarction, blocking cerebral blood vessels.

Cerebral infarction is characterized by the sudden appearance of neurological symptoms, and it is often found that the symptoms have developed overnight upon waking in the morning even though the patient felt fine before going to bed the previous night. Possible neurological symptoms include paralysis of the limbs, sensory abnormalities, speech disorders (aphasia, dysarthria), changes in consciousness, fainting, convulsions (epilepsy, seizures), walking disorders, urination disorders, headaches, visual disorders, facial paralysis, dysphagia (difficulty swallowing food), dementia (senility or cognitive decline), vomiting, dizziness, and ataxia.

### SUMMARY

An object of the present invention is to provide a pharmaceutical composition for preventing or treating cerebral infarction.

Another object of the present invention is to provide a health functional food for preventing or improving cerebral infarction.
1. A pharmaceutical composition for preventing or treating cerebral infarction, including micro RNA-382-5P (miR-382-5P).
2. The pharmaceutical composition for preventing or treating cerebral infarction according to the above 1, wherein the miR-382-5P includes a nucleotide sequence of SEQ ID NO: 1.
3. The pharmaceutical composition for preventing or treating cerebral infarction according to the above 1, wherein the cerebral infarction is cerebral thrombosis, cerebral embolism or lacunar cerebral infarction.
4. A health functional food for preventing or improving cerebral infarction, including micro RNA-382-5P (miR-382-5P).
5. The health functional food for preventing or improving cerebral infarction according to the above 4, wherein the miR-382-5P includes a nucleotide sequence of SEQ ID NO: 1.
6. The health functional food for preventing or improving cerebral infarction according to the above 4, wherein the cerebral infarction is cerebral thrombosis, cerebral embolism or lacunar cerebral infarction.

The pharmaceutical composition of the present invention has an effect of preventing or treating cerebral infarction.

The health functional food of the present invention has an effect of preventing or improving cerebral infarction.

The pharmaceutical composition and health functional food of the present invention have an effect of increasing the survival ability of nerve cells under cerebral ischemia conditions.

The pharmaceutical composition and health functional food of the present invention have effects of preventing cell death and promoting nerve cell regeneration.

### BRIEF DESCRIPTION OF THE DRAWINGS

FIG. 1 shows the results of Next Generation Sequencing (NGS) analysis on exosomes (NI) from stem cells induced by neural differentiation and exosomes (CON) from normal stem cells.

FIG. 2 shows the results of Gene Ontology (GO) analysis on hsa-miR-382-5P.

FIG. 3 shows the results of Kyoto Encyclopedia of Genes and Genomes (KEGG) analysis on hsa-miR-382-5P.

FIG. 4 is a photograph confirming the survival rate of neural cells (HT22) treated with hsa-miR-382-5P in oxygen-glucose deprivation (OGD) medium.

FIG. 5 is a graph showing the survival rate of neural cells (HT22) treated with hsa-miR-382-5P in oxygen-glucose deprivation (OGD) medium.

FIG. 6 is a schematic diagram illustrating the Wnt/β-catenin pathway.

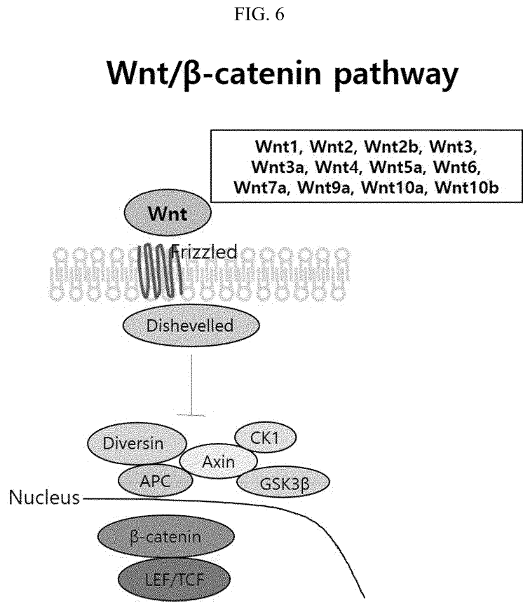

FIG. 7 is a graph showing the change in body weight over time in an animal model of cerebral infarction (MCAO) treated with hsa-miR-382-5P.

FIG. 8 is a graph showing the degree of improvement in behavior (use of forelimb) over time in an animal model of cerebral infarction (MCAO) treated with hsa-miR-382-5P.

FIG. 9 is a graph showing the degree of improvement in behavior (left-biased swing) over time in an animal model of cerebral infarction (MCAO) treated with hsa-miR-382-5P, as evaluated by the swing test.

FIG. 10 shows a brain image after hsa-miR-382-5P treatment in the MCAO model, with the range of brain lesions indicated by a white band.

FIG. 11 is a graph showing the extent of cerebral infarction measured by extracting the brain from an animal model of cerebral infarction (MCAO) treated with hsa-miR-382-5P.

### DETAILED DESCRIPTION

Hereinafter, the present invention will be described in detail.

The present invention relates to a pharmaceutical composition and a health functional food for preventing or treating cerebral infarction, which may exhibit a therapeutic effect on cerebral infarction by including micro RNA-382-5P (miR-382-5P).

In the miR-382-5P (miRBase accession number MI0000790), “mi” denotes “microRNA,” “R” denotes “mature miRNA,” “382” denotes a unique number (order of discovery), and “5P” means that it is generated from the antisense strand of the pri-miRNA that generates the “mature miRNA.”

The term “miRNA,” “microRNA” or “miR” refers to an RNA (or RNA analogue) that includes an endogenous, non-coding gene product whose precursor RNA transcript can form a small stem-loop, from which a mature “miRNA” is cleaved by the endonuclease Dicer. miRNAs are encoded within genes distinct from the mRNAs whose expression they regulate. The “miRNA,” “microRNA” or “miR” refers to a single-stranded RNA molecule of at least 10 nucleotides and no more than 35 nucleotides covalently linked together. The miRNA or complementary strand molecules thereof may include, but are not limited to, 15 to 50, 15 to 30, or 18 to 25 nucleic acids.

Cerebral infarction is a type of stroke that occurs when a blood vessel in the brain is blocked and the brain tissue supplied by the vessel becomes necrotic.

Cerebral infarction may be classified into cerebral thrombosis, cerebral embolism, and lacunar cerebral infarction. Cerebral thrombosis occurs when a blood clot forms in a cerebral blood vessel damaged by arteriosclerosis, thereby narrowing and blocking the blood vessel. Cerebral embolism occurs when a blood clot formed in a large artery of the heart or neck breaks off and flows through the bloodstream to block a distant cerebral blood vessel. Lacunar cerebral infarction refers to cerebral infarction caused by the blockage of a very small blood vessel in the brain.

The miR-382-5P may include the sequence GAAGUUGUUCGUGGUGGAUUCG (5′->3′, SEQ ID NO: 1).

The miR-382-5P sequence may be a sequence derived from the human genome or a miRNA sequence derived from the genome of another animal.

The miRNA of the present invention may include not only a miRNA represented by a specific nucleotide sequence (or sequence number) of the miR-382-5P, but also a precursor (pre-miRNA, pri-miRNA) of the miRNA, a miRNA having a biological function equivalent to that of the miR-382-5P, for example, a homolog (e.g., an ortholog), variants such as genetic polymorphisms, and derivatives thereof.

The miR-382-5P may be synthesized by a method known in the art or obtained by isolating it from cells. For example, when miR-382-5P is isolated and obtained from cells, miR-382-5P is contained in large quantities in exosomes of neural cells. Therefore, miR-382-5P can be isolated from exosomes of neural cells by a method known in the art, but is not limited thereto.

“Prevention” means any act of inhibiting or delaying the onset of cerebral infarction by administering a composition and/or health functional food.

“Treatment” or “improvement” means any act of improving or beneficially changing the symptoms of cerebral infarction by administering the composition and/or health functional food of the present invention. Anyone with ordinary knowledge in the technical field to which the present invention belongs will be able to know the exact criteria for the diseases to which the composition of the present invention is effective with reference to the materials presented by the Korean Medical Association, etc., and thus to determine the degree of improvement, enhancement, and treatment.

The composition of the present invention may include a pharmaceutically acceptable carrier. The pharmaceutically acceptable carrier refers to a pharmaceutically acceptable substance or vehicle, such as a liquid or solid filler, diluent, excipient, or solvent, which serves to transport an active ingredient from one organ or part of the body to another organ or part of the body.

The carrier may include, but is not limited to, saline, buffered saline, water, glycerol, and ethanol, and any suitable agent known in the art may be used.

The composition of the present invention may be administered orally or parenterally, and any administration method known in the art that allows the composition to reach a lesion where cerebral ischemia has occurred may be used without limitation.

The term “administration” as used herein means introducing the pharmaceutical composition of the present invention to a subject by any suitable method.

Parenteral administration methods may include, but are not limited to, intravenous, intramuscular, intraarterial, intramedullary, intrathecal, intracardiac, transdermal, subcutaneous, intraperitoneal, intranasal, enteral, topical, sublingual, or intrarectal administration, and are preferably administered into a cerebral blood vessel, more preferably into a vascular lesion where cerebral ischemia has occurred. For example, the parenteral administration methods may include directly injecting into a lesion where cerebral ischemia has occurred, or delivering to the lesion by injecting into an artery or vein, but are not limited thereto.

In the case of preparations for parenteral administration, they may be formulated in the form of injections, creams, lotions, ointments for external use, oils, moisturizers, gels, aerosols, and nasal inhalers using any method known in the art.

In the case of preparations for oral administration, the composition of the present invention may be formulated into powders, granules, tablets, pills, dragees, capsules, liquids, gels, syrups, slurries, or suspensions using any method known in the art. For example, oral preparations may be obtained by mixing an active ingredient with a solid excipient, grinding the mixture, adding a suitable adjuvant, and then processing the mixture into a granule to obtain a tablet or dragee. Examples of suitable excipients may include sugars such as lactose, dextrose, sucrose, sorbitol, mannitol, xylitol, erythritol, and maltitol; starches such as corn starch, wheat starch, rice starch, and potato starch; celluloses such as cellulose, methyl cellulose, sodium carboxymethyl cellulose, and hydroxypropylmethyl cellulose; and fillers such as gelatin and polyvinylpyrrolidone. Further, cross-linked polyvinylpyrrolidone, agar, alginic acid, or sodium alginate may be added as a disintegrating agent depending on the case. Furthermore, the composition of the present invention may additionally include anti-caking agents, lubricants, wetting agents, fragrances, emulsifiers, and preservatives.

The total daily dosage of the formulation of the present invention may be determined by a treating physician within the scope of correct medical judgment. A specific therapeutically effective amount for a given subject is preferably applied differently depending on various factors including the type and degree of the response to be achieved, the specific composition including whether other formulations are used, the age, weight, general health condition, sex, and diet of the subject, the time of administration, the route of administration, the secretion rate of the composition, the treatment period, drugs used together or simultaneously with the specific composition, and similar factors well known in the medical field. Therefore, the preferable dosage of the composition of the present invention may be determined in consideration of the above-mentioned matters, and may be appropriately selected by a person skilled in the art.

The composition of the present invention may be used alone for the prevention or treatment of cerebral infarction, or may be used in combination with methods such as surgery, hormone therapy, drug therapy, and biological response modifiers.

The subject to which the composition of the present invention can be applied may be any animal capable of developing such a disease, and the subject may include not only humans and primates, but also livestock such as cows, pigs, sheep, horses, dogs, and cats.

Further, the present invention provides a health functional food for preventing or improving cerebral infarction including micro RNA-382-5P (miR-382-5P).

The miR-382-5P and cerebral infarction are as described above.

The health functional food of the present invention may include a nutritionally acceptable carrier. The nutritionally acceptable carrier refers to a pharmaceutically acceptable substance, composition or vehicle, such as a liquid or solid filler, diluent, excipient or solvent, which serves to transport an active ingredient from one organ or part of the body to another organ or part of the body.

The health functional food of the present invention may be a beverage (including alcoholic beverages), fruit and processed foods thereof (e.g., canned fruit, bottled fruit, jam, marmalade, etc.), fish, meat and processed foods thereof (e.g., ham, sausage, corned beef, etc.), bread and noodles (e.g., udon, buckwheat noodles, ramen, spaghetti, macaroni, etc.), fruit juice, various drinks, cookies, taffy, dairy products (e.g., butter, cheese, etc.), edible vegetable oil, margarine, vegetable protein, retort food, frozen food, and various seasonings (e.g., soybean paste, soy sauce, sauces, etc.), which include the miR-382-5P.

Further, the health functional food of the present invention may be formulated as a tablet, pill, powder, granule, powder preparation, capsule, liquid formulation, etc. They may be formulated by further including one or more of a carrier, a diluent, an excipient, and an additive.

Additives that can be further included in the health functional food of the present invention may include one or more ingredients selected from the group consisting of natural carbohydrates, flavoring agents, nutrients, vitamins, minerals (electrolytes), flavoring agents (synthetic flavoring agents, natural flavoring agents, etc.), coloring agents, fillers (cheese, chocolate, etc.), pectic acid and its salts, alginic acid and its salts, organic acids, protective colloid thickeners, pH regulators, stabilizers, preservatives, antioxidants, glycerin, alcohol, carbonating agents, and fruit pulp.

In addition to the above, the health functional food of the present invention may contain various nutrients, vitamins, minerals (electrolytes), flavoring agents such as synthetic flavoring agents and natural flavoring agents, coloring agents and fillers (cheese, chocolate, etc.), pectic acid and its salts, alginic acid and its salts, organic acids, protective colloid thickeners, pH regulators, stabilizers, preservatives, glycerin, alcohol, carbonating agents used in carbonated beverages, etc. Further, the health functional food of the present invention may contain fruit pulp for the production of natural fruit juice and vegetable beverages. These ingredients may be used independently or in combination.

Specific examples of the carriers, excipients, diluents, and additives may include lactose, dextrose, sucrose, sorbitol, mannitol, erythritol, starch, acacia gum, calcium phosphate, alginate, gelatin, calcium silicate, microcrystalline cellulose, polyvinylpyrrolidone, cellulose, methylcellulose, water, sugar syrup, methyl hydroxybenzoate, propyl hydroxybenzoate, talc, magnesium stearate, mineral oil, and mixtures thereof, but are not limited thereto.

The content of the health functional food of the present invention as an effective ingredient in the above-described formulation may be appropriately adjusted depending on the form and purpose of use, the user's condition, the type and severity of symptoms, etc., and may be 0.001 to 99.9% by weight (“wt %”), preferably 0.01 to 50 wt %, based on the solid weight, but is not limited thereto.

Hereinafter, the present invention will be described in detail by way of examples.

### EXAMPLE

1. Next Generation Sequencing Analysis of Exosomes from Stem Cells Induced to Undergo Neural Differentiation

Small RNA sequencing of exosomes from cells differentiated from stem cells into neural cells was performed using a Multiplex Small Library Prep kit, and miRNAs effective in treating intractable diseases were selected.

The analysis was conducted with exosomes obtained from normal stem cells (CON) and exosomes differentiated into neural cells (NI). NGS selection criteria were (fold change: 10, normalized data (log 2): 10, p-value: 0.05).

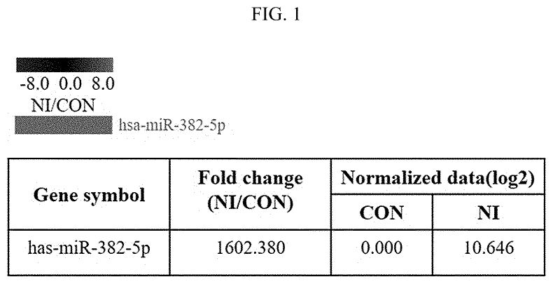

The screening results showed that hsa-miR-382-5P had a value of 1602.280 in NI compared to CON, and the log 2 value was 0.000 in CON and 10.646 in NI, indicating that miRNAs not present in CON but present only in NI were selected (FIG. 1).

2. Gene Ontology Analysis of hsa-miR-382-5P

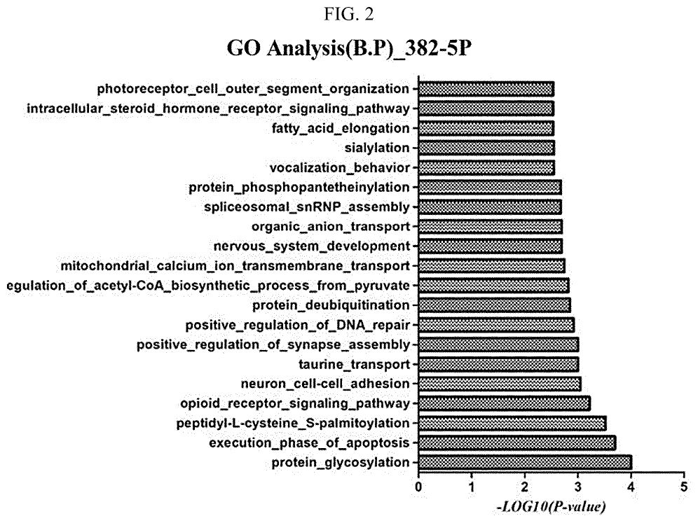

After selecting 20 gene categories as shown in the y-axis list of FIG. 2, they were re-selected with a focus on mechanisms involved in the treatment of intractable diseases.

When converted to log 10, neuron cell-cell adhesion was confirmed to be 3.045, 2.69 for nervous system development, and 2.552 for vocalization behavior.

Since it is involved in neuron cell-cell adhesion, nervous system development, and vocalization behavior, this suggests that it has therapeutic significance for intractable diseases such as stroke, dementia, and Parkinson's disease.

3. Kyoto Encyclopedia of Genes and Genomes (KEGG) Analysis of hsa-miR-382-5P

Analysis of hsa-miR-382-5P using Kyoto Encyclopedia of Genes and Genomes (KEGG) data confirmed that it is involved in the neurotrophin signaling pathway, dopaminergic synapse, PI3K-AKT signaling pathway, and apoptosis.

Neurotrophins are involved in the survival and differentiation of neurons. Since they are growth factors that promote neuronal survival, they are associated with intractable diseases such as stroke, dementia, and Parkinson's disease.

The PI3K-AKT signaling pathway is known to be a mechanism involved in neurological diseases and intractable diseases.

Since intractable diseases are associated with cell death, hsa-miR-382-5P has therapeutic effects in intractable diseases.

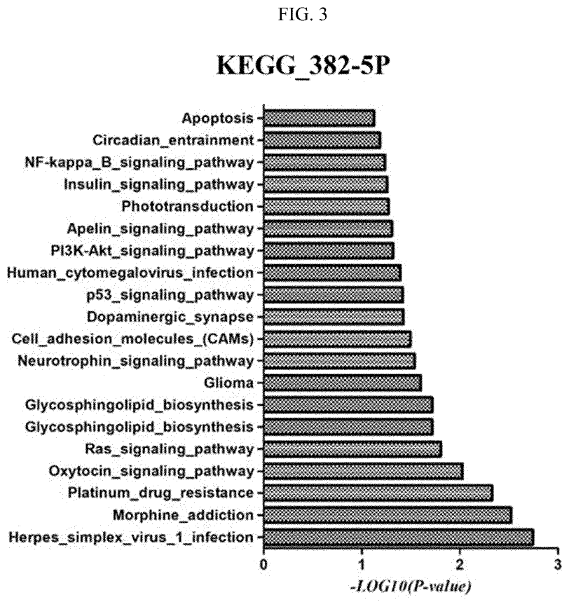

After selecting 20 gene categories as shown in the y-axis list of FIG. 3, they were re-selected with a focus on mechanisms involved in intractable disease treatment.

After log 10 conversion, the values were 1.54 for the neurotrophin signaling pathway, 1.32 for the PI3K-AKT signaling pathway, 1.43 for the dopaminergic synapse, and 1.13 for apoptosis (FIG. 3).

4. Effect of hsa-miR-382-5P on Oxygen-Glucose Deprivation (OGD)

Oxygen-glucose deprivation (OGD) and reoxygenation, which are in vitro experimental methods of cerebral ischemia, were performed. N₂ was used to block oxygen, and cerebral ischemia conditions were induced in the cells using a culture medium from which FBS and glucose were removed. OGD experiments were performed for 12 hours using the FBS- and glucose-deprived culture medium under environmental conditions of 1% N₂, 5% CO₂, and 37° C. After 12 hours, hsa-miR-382-5P was added to the medium at 10 μmol. A comparative analysis was conducted between the group treated with hsa-miR-382-5P and the untreated control group. A statistically significant difference was found between the two groups. Cell viability was measured as 32.50±3.981% in the control group and 52.87±1.262% in the hsa-miR-382-5P group (p<0.005).

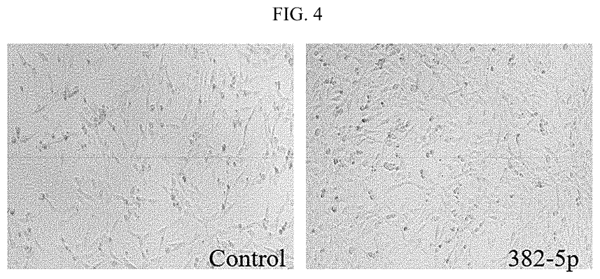

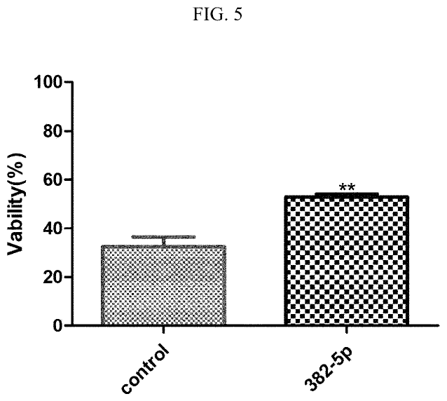

As a result, hsa-miR-382-5P was suggested to be therapeutically effective in ischemic stroke (FIGS. 4 and 5).

### 5. Wnt Antibody Array

After OGD treatment of HT22 cells, proteins were extracted from HT22 cells, and then a Wnt array was performed using a Fullmoon Wnt antibody array slide. The results are shown in Table 1.

Since Wnt is involved in apoptosis, proliferation and migration, a Wnt antibody array was performed. A comparative analysis was conducted between the group treated with hsa-miR-382-5P and the untreated group. The activity and comparative values of markers existing in the Wnt/β-catenin pathway, which is a canonical Wnt pathway, were selected. As a result of the comparative analysis between the group treated with hsa-miR-382-5P and the untreated group in Wnt1 and Wnt5a, which are involved in the Wnt/β-catenin pathway, the fold change values were 1.262 for Wnt1 and 1.123 for Wnt5a.

Further, the values of GSK3-β and APC involved in the Wnt/β-catenin pathway were confirmed. As a result of comparative analysis between the group treated with hsa-miR-382-5P and the untreated group, the fold change values were 0.970 for GSK3-β and 1.262 for APC. Through the activity of these markers, it was confirmed that the Wnt/β-catenin pathway was activated, and through this mechanism, the activity of β-catenin in the nucleus was also confirmed.

As a result of comparative analysis between the group treated with hsa-miR-382-5P and the untreated group, the fold change value of β-catenin was 0.976. Consequently, it was confirmed that in the group treated with hsa-miR-382-5P, genes involved in the canonical Wnt pathway related to apoptosis and nerve regeneration were expressed. Therefore, it was confirmed that hsa-miR-382-5P is an effective substance for the treatment of ischemic stroke, as it is involved in nerve regeneration and apoptosis.

TABLE 1

| Gene | Fold change |
|---|---|
| Wnt1 | 1.262 |
| Wnt5a | 1.123 |
| GSK3-β | 0.970 |
| APC | 1.262 |
| β-catenin | 0.976 |

6. Effect of hsa-miR-382-5P in a Cerebral Infarction Model (MCAO)

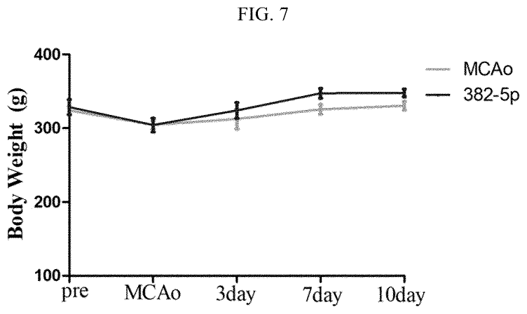

A cerebral infarction animal model was created using the middle cerebral artery occlusion (MCAO) experimental method. Ten-week-old SD rats were used, and the experiment was conducted under sodium pentobarbital anesthesia. The blood flow at the base of the middle cerebral artery was blocked for 1 hour using a 17 mm probe, after which the probe was removed. The induction of cerebral infarction was confirmed by weight loss and behavioral tests of the animals. Seven days later, animals with induced cerebral infarction (MCAO) were injected intracerebroventricularly with 20 nmol of hsa-miR-382-5P using stereotaxic surgery. Thereafter, behavioral analysis was performed to evaluate the therapeutic effect on cerebral infarction. After MCAO induction, the body weight of the animal model temporarily decreased, but it was confirmed to increase when hsa-miR-382-5P was injected (FIG. 7).

### 1) Experiment to Analyze Forelimb Usage Rate in the MCAO Animal Model

Since temporary ischemia was induced in the left blood vessel, animals with cerebral infarction had difficulty using the right forelimb. Therefore, the forelimb usage rate was measured in the animal model. Behavioral analysis was performed for 10 days after drug injection.

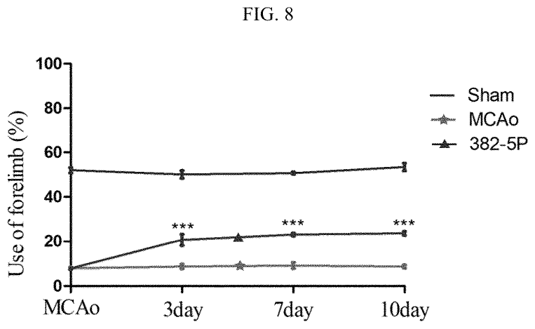

The test results showed that the model injected with hsa-miR-382-5P demonstrated improved behavior on the third day after injection (FIG. 8). The forelimb usage rate (%) was 8.62±0.87% in the MCAO control group and 19.43±3.14% in the hsa-miR-382-5P injected group on day 3 after hsa-miR-382-5P injection (p<0.0005). On day 7 after hsa-miR-382-5P injection, the MCAO control group was 9.56±1.23% and the hsa-miR-382-5P injected group was 22.63±0.86% (p<0.0005). On day 10 after hsa-miR-382-5P injection, the MCAO control group was 9.17±0.84% and the hsa-miR-382-5P injected group was 22.44±0.19% (p<0.0005).

As a result, the recovery rate was higher in the group injected with hsa-miR-382-5P in the MCAO model, suggesting that hsa-miR-382-5P may be used as a therapeutic agent for cerebral infarction.

### 2) Analysis of Left-Turning Behavior in the MCAO Animal Model

The therapeutic effect of hsa-miR-382-5P on cerebral infarction was evaluated using the swing test.

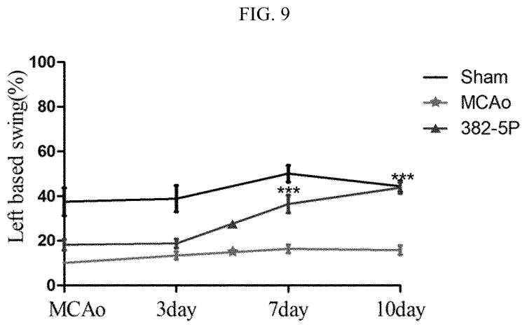

The test results showed that the model injected with hsa-miR-382-5P demonstrated improved behavior on day 7 after injection (FIG. 9). The behavioral percentage of leftward turning was 13.33±1.67% in the MCAO control group and 18.75±2.10% in the hsa-miR-382-5P injected group on day 3 after hsa-miR-382-5P injection (p=NS). On day 7 after hsa-miR-382-5P injection, the MCAO control group was 16.25±1.83%, while the hsa-miR-382-5P group was 36.48±3.92% (p<0.0005). On day 10 after hsa-miR-382-5P injection, the MCAO control group was 15.71±2.02%, while the hsa-miR-382-5P injected group was 43.75±2.46% (p<0.0005).

As a result, the recovery rate was higher in the group injected with hsa-miR-382-5P in the MCAO model, suggesting that hsa-miR-382-5P may be used as a therapeutic agent for cerebral infarction.

### 3) Measurement of Brain Lesion Extent

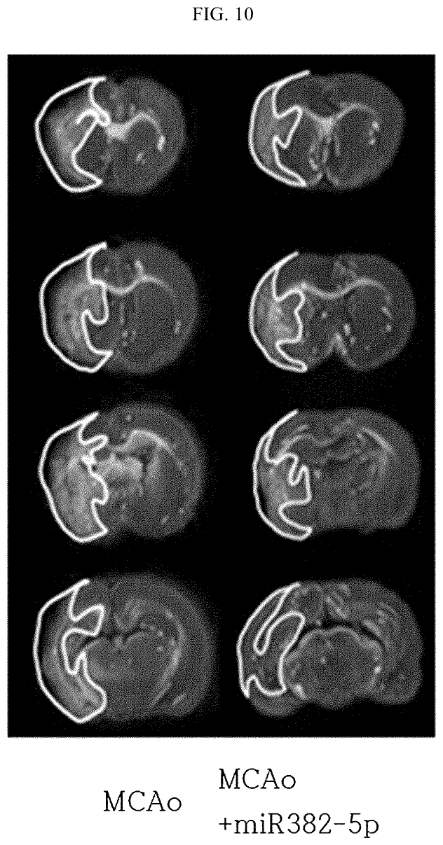

To confirm whether brain lesions were reduced after hsa-miR-382-5P injection, the triphenyltetrazolium chloride (TTC) test was performed. After administering sodium pentobarbital anesthesia to the animal model, the brain was excised. The excised brain was stained using TTC solution, and the lesion area was stained white. It was confirmed that the animal model injected with hsa-miR-382-5P showed an improvement in cerebral infarction (FIG. 10).

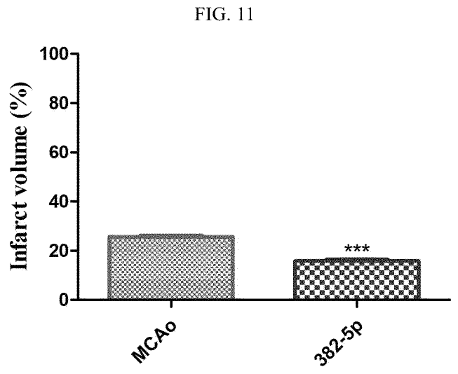

When the brain of the animal model was excised 10 days after hsa-miR-382-5P injection and the infarction extent was measured, the MCAO control group was 25.64±0.55%, while the hsa-miR-382-5P injected group was 15.88=0.69% (p<0.0005) (FIG. 11).

This suggests that hsa-miR-382-5P injection reduced the infarction extent, and consequently, it may be used as a therapeutic agent for cerebral infarction.

1-6. (canceled)

7. A method for treating cerebral infarction, the method comprising:
administering a composition comprising micro RNA-382-5P (miR-382-5P) to a subject in need thereof.

8. The method of claim 7, wherein the miR-382-5P comprises a nucleotide sequence of SEQ ID NO: 1.

9. The method of claim 7, wherein the cerebral infarction is cerebral thrombosis, cerebral embolism and/or lacunar cerebral infarction.

10. The method of claim 7, wherein the cerebral infarction is cerebral thrombosis.

11. The method of claim 7, wherein the cerebral infarction is cerebral embolism.

12. The method of claim 7, wherein the cerebral infarction is lacunar cerebral infarction.

13. The method of claim 7, wherein the composition is a pharmaceutical composition comprising the micro RNA-382-5P (miR-382-5P) and a pharmaceutically acceptable carrier.

14. The method of claim 7, wherein the composition is a functional food comprising the micro RNA-382-5P (miR-382-5P) and a nutritionally acceptable carrier.


SEQUENCE LISTING (1 sequence)

SEQ ID NO: 1 - RNA, 22 nt, Homo sapiens
  source 1..22: mol_type = other RNA
gaagttgttcgtggtggattcg